(19) United States
(12) Patent Application Publication
(10) Pub. No.: US 2010/0196194 A1
(43) Pub. Date: Aug. 5, 2010

(54) METHOD FOR STERILIZING OBJECTS WITH OZONE

(75) Inventors: Rudolphus Gerardus Henricus Maria Voeten, Wouw (NL); Gustaaf Ludovicus Andreas Rejinders, Stampersgat (NL); Silvester Karel Marinus Johannes van Voorthuisen, Rijsbergen (NL)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(73) Assignee: BRADFORD INSTRUMENTS BV, TG Heerle (NL)

(21) Appl. No.: 12/514,185

(22) PCT Filed: Nov. 7, 2007
(86) PCT No.: PCT/NL2007/000278

§ 371 Date: Apr. 7, 2010

(30) Foreign Application Priority Data
Nov. 8, 2006 (NL) 1032835

(51) Int. Cl. A61L 2/20; A61L 2/24

(52) U.S. Cl. 422/3

(57) ABSTRACT

A method for sterilizing objects with ozone comprises the steps of: positioning the objects in a sterilization chamber (10); effecting an ozone-containing gas atmosphere in the sterilization chamber (10); making the ozone-containing gas circulate via a circulation pipe (20) outside the sterilization room (10), wherein the circulation pipe (20) is provided with an ozone generator (60) and a water mouthpiece (73); establishing an air humidity in a range of approximately 80%; establishing an ozone concentration in the gas higher than 0.2 mgr/l; defining in advance an ozone performance equivalent (OPEQ); measuring the ozone concentration (OC) during the sterilization process on certain measurement moments; calculating the time integral of the measured ozone concentration; wherein the sterilization process is continued until the time integral of the measured ozone concentration is minimally equal to the predetermined ozone performance equivalent.

Generally, the present invention relates to sterilizing objects, such that they are medically “clean”, by which is meant that all micro-organisms possibly present are killed.

In practice, bacteria have proven relatively easy to kill, but the killing of bacterial spores, which is at least equally important for a good sterilization, is much more difficult. It is therefore an objective of the present invention to provide a sterilization method which also eliminates bacterial spores with great certainty.

Methods for sterilizing instruments are known per se. In those methods, the instruments are exposed for some time to an atmosphere that is lethal to micro-organisms. Conventionally, steam was used for this purpose, but that requires very high temperatures. An effectively sterilizing gas that could be used at lower temperatures is ethylene oxide. The instruments to be sterilized are placed in a sterilization chamber, that was subsequently evacuated and filled with ethylene oxide. The chamber thus filled with ethylene oxide is left alone during a predetermined process time; afterwards, the ethylene oxide is sucked away, the sterilization chamber is ventilated, and the instruments are taken out of the chamber. This process is a batch process.

A problem with ethylene oxide is that it is very toxic and very difficult to decompose. This means that degassing the sterilization chamber after the processing time takes very long. Furthermore, it is necessary that the sterilization apparatus is surrounded with strict safety precautions. To bring relief to this, a replacing gas has been searched, and that was found in the form of ozone, which derives its effectiveness from its strongly oxidizing property. Further, the process was executed as described above, on the understanding that the ethylene oxide was replaced by ozone. In practice, it turned out that the sterilization process was not going well enough then, and it was found that, for killing spores, it was necessary to raise the degree of humidity in the atmosphere to approximately 95%. An example of this technology is described in WO-00/66186.

Such high humidity, however, also entails disadvantages. An important problem is the occurrence of condensation on the objects to be sterilized. The moisture forms, as it were, a covering film over the objects, causing the surface of the relevant objects to be less easily accessible for the ozone and thus the sterilization process to be less effective.

Furthermore, in the equipment, the chance on condensation and corrosion is present, so the equipment either has to be specially designed for corrosion resistance or needs to be inspected regularly and repaired if needed. Furthermore, condensation means there can be places in the apparatus where moisture keeps standing, which thus are potentially favorable growing circumstances for bacteria and fungi.

There are roughly two ways for providing ozone to a sterilization chamber: ozone is provided in a gas bottle, or ozone is created from oxygen, wherein oxygen can be obtained from a gas bottle or from the atmosphere. Converting oxygen from the ambient air to ozone is preferred, but also in this context the high degree of air humidity is a problem: converting oxygen to ozone is more difficult, while the ozone can also react with moisture. When, during the sterilization process, ozone reacts with moisture, the ozone concentration drops and thus the effectiveness of the sterilization process will decrease. When a fixed process time is maintained, then the chance exists that the sterilization is incomplete. Conversely, the process time can be chosen so long that also in case of a less effective sterilization process the sterilization will be complete nevertheless, but this implies that the sterilization process is continued unnecessarily long in cases where the sterilization process is completely effective.

Normally, an ozone generator working on the basis of a corona discharge is used for generating ozone from oxygen. In the case of such an ozone generator, cooling is needed, because otherwise the degradation of ozone to oxygen is accelerated. In the case of the device as described in said publication WO-00/66186, use is made of an external cooling, which increases the complexity and costs of the apparatus.

The said publication WO-00/66186 describes the necessity to work with huge quantities of ozone: the publication mentions quantities from 48-96 mgr/l. To reach this, a large ozone generator with a large capacity is needed; the publication even mentions the presence of two generators in parallel. Furthermore, for the benefit of the ozone generation, the required oxygen needs to be provided in pure form and it is not sufficient to use oxygen from the environment.

Furthermore, the said publication WO-00/66186 describes the necessity to perform the process at strongly reduced pressure in order to reduce the condensation problems, but this requires the presence of vacuum equipment.

The present invention aims at solving the problems mentioned, at least at minimizing.

More in particular, the present invention aims at providing an efficient and reproducible sterilization process, as well as a relatively simple apparatus for executing the process.

For an important part, the present invention is based on the insight that it is not necessary to sterilize with high ozone and moisture concentrations, but that an effective sterilization process can be reached at more moderate ozone and moisture concentrations and at approximate atmospheric conditions. By the presence of moisture in moderate quantities of approximately 60-80%, the operation of the ozone is positively influenced without the drawbacks of condensation becoming strong. Thus, there are no complicated measures needed to fight the condensation problems, it is possible to generate ozone from the ambient air, and the ozone generator can be a relatively small generator, so that the complexity and costs of the required device are strongly reduced.

According to a further important aspect of the present invention, it is furthermore not required that the ozone concentration and the moisture content are controlled on values set beforehand. Experiments have demonstrated that it is possible to operate the ozone generator and a moisture generator in an uncontrolled modus, meaning that they are simply turned “ON”, wherein then, during the process, an equilibrium situation sets itself with an ozone concentration and a moisture content depending on the circumstances, wherein especially the momentary ambient temperature plays a large role as variable factor. Furthermore, of course, the production capacities of the ozone generator and the moisture generator in relation to the dimensions of the sterilization chamber are important as constant factors. In an experimental set-up, good results have been reached at an ozone concentration of 2-3 mgr/l (based on 1 atm) and a moisture content in the range of 60-80%.

According to a further important aspect of the present invention, a sterilization device comprises a gas circulation loop of which the sterilization chamber and the ozone generator are part. During the sterilization process, the gas is continuously circulated through the gas circulation loop, at a fairly high velocity. Thereby, several advantages are offered at the same time. The high gas flow velocity supplies a cooling for the ozone generator. Furthermore, the continuous gas flow in the sterilization chamber offers the advantage of keeping the ozone concentration in the sterilization chamber better homogeneous, and also of difficult accessible places receiving sufficient ozone.

Furthermore, by incorporating an ozone sensor in the gas circulation loop, the ozone concentration in the sterilization chamber can be guarded.

According to a further important aspect of the present invention, possible fluctuations in the ozone concentration are compensated by variations in the treatment time. Experiments have demonstrated that also at lower ozone concentration an effective sterilization is possible, on the understanding that a longer treatment time is needed then. It has turned out that a 100% sterilization is reached if the product of ozone concentration and treatment time reaches a minimum value (to be determined experimentally beforehand), which will be indicated as the ozone performance equivalent. At a constant ozone concentration, this means that the sterilization process may be stopped as soon as the treatment time is equal to the ozone performance equivalent divided by the ozone concentration; of course, continuing longer is allowed, but it is of no more use then. More in general, therefore, the present invention proposes to measure the instantaneous ozone concentration and to calculate the time integral thereof; the sterilization process may be stopped then as soon as this time integral is equal to the ozone performance equivalent. Periods of lower ozone concentrations, either being short or long, translate themselves to a longer treatment time without the risk of an incomplete sterilization.

These and other aspects, characteristics and advantages of the present invention will be further explained by the following description with reference to the drawings, in which equal reference numbers refer to equal or comparable parts, and in which:

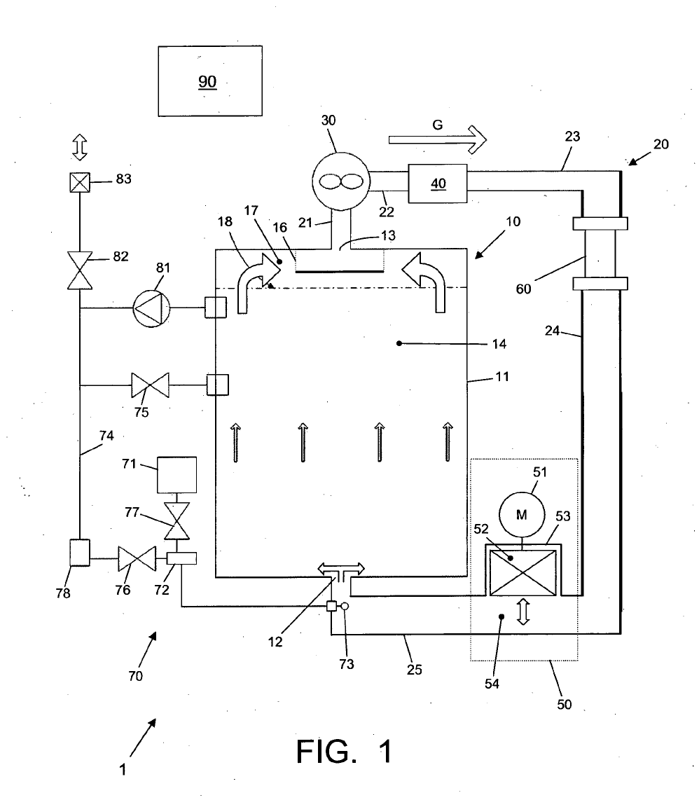

FIG. 1 schematically illustrates a sterilization apparatus according to the present invention;

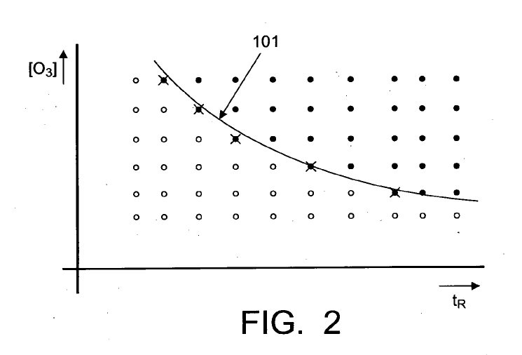

FIG. 2 is a graph that illustrates measurements with relation to sterilization;

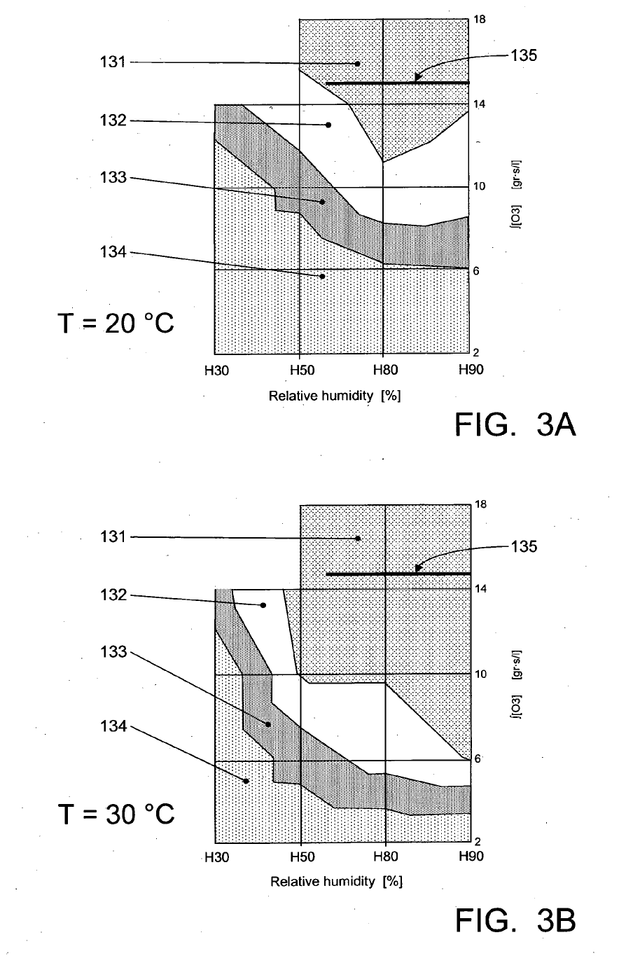

FIG. 3A-B are graphs that show a relationship between ozone performance equivalent and degree of humidity;

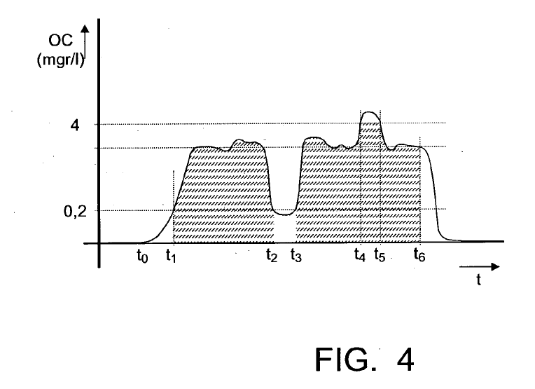

FIG. 4 is a graph that schematically shows a possible course of the ozone concentration as function of the time during a sterilization process;

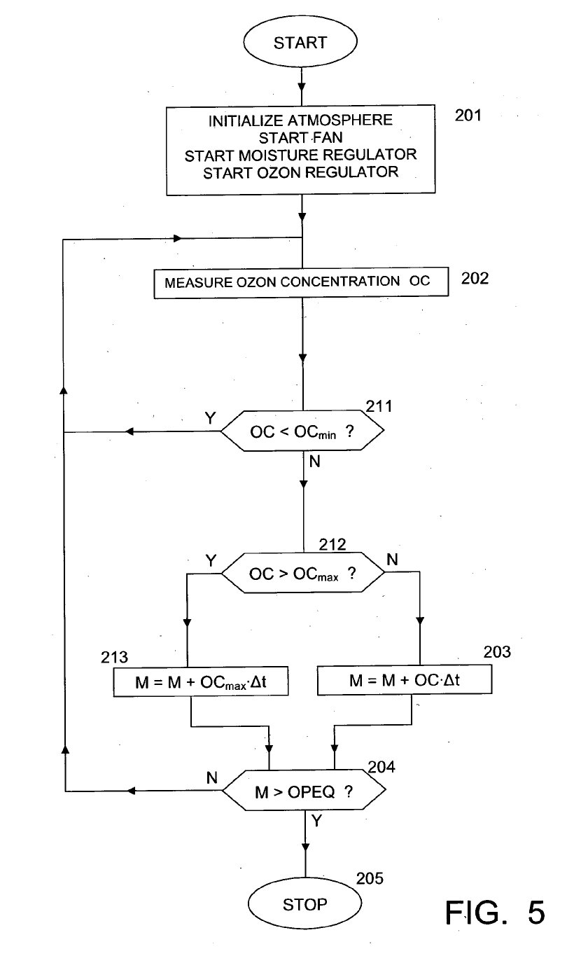

FIG. 5 is a flow diagram that illustrates steps of a sterilization process according to the present invention.

FIG. 1 is a block diagram that illustrates the general design of a sterilization apparatus 1 according to the present invention. The apparatus 1 has a sterilization chamber 10, in which objects to be sterilized (not shown for the sake of simplicity) can be placed. The chamber 10 has a wall 11, with at least one door therein for placing and taking away the objects to be sterilized (which door for the sake of simplicity is not shown either). The wall 11 has a gas inlet opening 12 and a gas outlet opening 13. A circulation pipe 20 is connected to these openings.

A circulating gas flow G is maintained through the circulation pipe 20 and the chamber 10 by a fan 30. In the sketched example, the fan 30 is arranged directly after the gas outlet opening, wherein a first pipe section 21 of the circulation pipe 20 connects the gas outlet opening 13 of de chamber 10 to an entrance of the fan 30. It is also possible that the fan 30 is mounted directly against the chamber 10, so that the first pipe section 21 can be left out.

Seen in flow direction, the gas circulation circuit comprises the following parts:

a sensor 40, wherein a second pipe section 22 connects an output of the fan 30 to an entrance of the sensor 40;

an ozone generator 60, wherein a third pipe section 23 connects an output of the sensor 40 to an entrance of the ozone generator 60;

an ozone destructor device 50, wherein a fourth pipe section 24 connects an output of the ozone generator 60 to an entrance of the destructor 50, while a fifth pipe section 25 connects an output of destructor 50 to the gas inlet opening of the chamber 10.

In relation to the ozone generator 60, it is noted that use can be made here of a usual ozone generator, operating according to the corona discharge principle, so a further description of the generator 60 can be omitted here. It is sufficient to note that an external cooling may be omitted, or may be implemented with decreased cooling capacity, in view of the cooling effect of the flowing gas.

In relation to the ozone destructor 50, it is noted that this serves to remove ozone from the gas mixture after termination of the sterilization process. During the sterilization process, the destructor 50 is not active. This is achieved because the destructor 50 has a destruction member 52 that can be positioned in and out of the gas flow, displaceable by a motor 51. During the sterilization process, the destruction member 52 is situated in the parking chamber 53 next to a gas flow channel 54, so that gas flowing in the gas flow channel 54 is not influenced by the destruction member 52. When the sterilization process is completed, the motor 51 is excited to move the destruction member 52 from the parking chamber 53 to a position in the gas flow channel 54. The gas flow is continued, and the gas flowing in the gas flow channel 54 is influenced by the destruction member 52, wherein ozone is intercepted and reduced to oxygen or an oxygen compound. Since for this purpose use can be made of ozone destruction materials and/or catalysts known perse, for example activated carbon and/or platinum, a further description of the destructor 50 can be omitted here.

Instead of a displaceable destruction member 52, the destructor 50 could have two parallel flow channels, wherein one of the channels leads by or through a destruction material while the other channel is free of destruction materials, wherein, for example by means of controllable valves, a choice is made to lead the gas flow through either the one or the other flow channel.

In relation to the sensor 40, it is noted that the term “sensor” is used here as a generic term, which can relate to a single detector as well as to a system of multiple detectors.

In the case of multiple detectors, it is possible that these detectors are positioned together in a common sensor casing, but that is not necessary: the detectors may be positioned independent from each other.

In any case, the sensor 40 comprises an ozone detector for measuring the ozone concentration of the gas. In the preferred embodiment, the ozone generator 60 is continuously on during the sterilization process, but if desired it is also possible that the measured ozone concentration is passed on to a control member 90, which switches the ozone generator 600N or OFF depending on the measured ozone concentration.

Furthermore, the sensor 40 may comprise a detector for measuring the degree of humidity, wherein the measuring result can be used for switching the moisture producing device 700N or OFF. Further, it is possible that the moisture producing device 70 operates independently, but it is also possible that the moisture producing device 70 is switched by the control member 90.

The moisture producing device 70 comprises a storage-vessel 71 for water, a pump 81, an auxiliary storage vessel 72, a pressure chamber 78 and a mouthpiece 73, which is mounted in the fifth pipe section 25, short before the gas inlet opening 12. The water can be delivered pulsating in the shape of vapor, or mist, or small droplets. Although ambient air can be used as propellant for the water vapour or mist, it is advantageous, for this purpose, to use gas from the sterilization chamber 10, to which a system of propelling pipe 74 with valves 75, 76, 77 serves.

Although it is conceivable that the order of the fan 30, sensor 40, destructor 50, generator 60 and moisture supply mouthpiece 73 is different, the shown and described order is preferred. Because the moisture supply mouthpiece 73 is situated downstream of the ozone generator 60, the just added moisture does not directly affect the operation of the ozone generator 60. Because the sensor 40 is situated between the output 13 of the chamber and the ozone generator 60, the sensor 40 measures at the location with the lowest ozone concentration in the system, which implicates that there is never less ozone in the chamber 10 than indicated by the sensor 40. Because the fan 30 is situated directly behind the output 13 of the chamber 10, a possible degradation of the ozone, stimulated by the fan 30, will have no influence on the sterilization process.

In relation to the chamber 10, the gas outlet'opening 13 is set up diametrically opposite the gas inlet opening 12 so that the gas is forced to cross the entire chamber 10. Further, the gas outlet opening 13 and the gas inlet opening 12 may be situated in a top wall and a bottom wall, so that the gas flow through chamber 10 is directed vertically, or the gas outlet opening 13 and the gas inlet opening 12 may be situated in side walls so that the gas flow through the chamber 10 is directed horizontally.

The chamber 10, following from the nature of the matter, is larger that the transverse dimension of the circulation pipe 20: the circulation pipe 20 may be implemented by, for example, a round tube with an inner diameter of approximately 5 cm, while characteristic dimensions of chamber 10 are typically in the order of 30 cm and more. As a consequence, the flow velocity of the gas in the chamber 10 is considerably lower than in the pipe 20. The fan 30 and the pipe 20 are chosen to enable a gas velocity of approximately 800 litre/minute, which at a pipe diameter of 5 cm corresponds to a flow velocity in the order of approximately 8 m/s while in a chamber with dimensions of 30×30×30 cm³ the flow velocity approximately amounts to 0.3 m/s.

To bring about a good distribution of the gas flow over the entire chamber 10, the chamber 10 is provided with a flow distributor 18 extending in front of the outlet opening 13 and in the shape of a perforated plates, a wire gauze or the like, as well as with a flow filter 16′ extending in front of the outlet opening 13 and having a substantially closed bottom and partially permeable walls between the edges of the flow filter 16 and the top wall of the chamber 10. Thereby the gas flow will be forced to pass the process compartment 14 over its entire width and entire height before bending, in a converging room behind the flow distributor 18, to the central output, as illustrated by means of two bent arrows. Thereby, and by the fact that the flow in the process compartment 14 is turbulent, the result is achieved that all objects to be sterilized present in the process compartment 14 are circumfluenced by gas in substantially the same way.

For safety reasons, in order to prevent ozone from ending up in the atmosphere, the sterilization apparatus 1 is preferably operated at a pressure in the chamber 10 that is lower than atmospheric pressure. For that purpose, an evacuation pump 81 is connected to the chamber 10, which, via a valve 82 and a filter 83, can suck away gas from the chamber 10 and blow this gas away to the environment. The filter 83 comprises an ozone filter and a bacteria filter (HEPA).

Experimentally, the relation has been investigated between ozone concentration and the time needed for sterilization. A typical measurement procedure is as follows. A sample with bacterial spores is prepared, and exposed in the chamber 10 to an ozone-containing atmosphere with a specific temperature and air humidity, during a specific treatment time, after which the sample is removed from the chamber 10. A comparable sample is prepared, and exposed in the measurement chamber to an ozone-containing atmosphere, wherein ozone concentration, temperature and air humidity are kept equal as much as possible; only the treatment time is chosen different. Thus, a series of samples is treated, each time with different treatment times. To obtain a greater certainty, the measurements are repeated, which means that at a certain treatment time always multiple measurements are done with different samples.

The treated samples are subsequently placed in a conditioned breeding chamber, and it is monitored at which samples bacterial growth is and at which samples bacterial is not taking place. If bacterial growth takes place, the sterilization was apparently insufficient; if no bacterial growth takes place, apparently all spores were killed.

FIG. 2 is a graph that schematically and in idealized way illustrates the measurement results. Here, the vertical axis represents the ozone concentration [O₃] in arbitrary units, and the horizontal axis represents the time t_{R} needed for sterilization in arbitrary units. Measurement points are indicated by circles.

The results obtained from the culture were correlated to the treatment times. For each value of the ozone concentration, it was assessed what the LONGEST treatment time was where bacterial growth was still observed: this treatment time was apparently insufficient to guarantee 100% elimination of the spores; this measurement point and all measurement points with shorter treatment times, in FIG. 2, are indicated with open circles. At the longer treatment times, apparently, each time all spores were eliminated. From these longer treatment times the SHORTEST was taken as the minimally needed treatment time t_{R}: these measurement points are indicated in FIG. 2 with a cross.

The procedure described above was executed at different values of the ozone concentration, wherein the other parameters were kept constant as well as possible, thus to obtain different points for the graph of FIG. 2.

In the present experiment, the ozone concentration was varied in the range from 2 mgr/l to 3 mgr/l; the measured minimally needed treatment time t_{R} proved to vary in the range from 90 min to 120 min.

The graph of FIG. 2 illustrates in a global way that at higher ozone concentrations a good sterilization can be reached in a relatively short time (top left in the graph), while at lower ozone concentrations a longer time is needed (bottom right in the graph).

In FIG. 2 furthermore is illustrated that it is possible to define a curve 101 that satisfies the equation OC·t=constant, wherein this constant is chosen such that this curve for not any ozone concentration has a time value lower than the measured minimally needed treatment time t_{R} at that ozone concentration. In other words, at each ozone concentration OC the approximating curve 101 shows a treatment time that is minimally equal to the minimally needed treatment time t_{R} at that ozone concentration. In FIG. 2, this is visually recognizable because there are closed measurement points to the left of the curve, but no open measurement points to the right of the curve.

The constant in the above-mentioned formula will hereafter be indicated as ozone performance equivalent OPEQ.

The FIGS. 3A-3B illustrate another experiment, executed on spores of the bacterium Bacillus Atrophaeus (previously known as Bacillus subtilis var. niger); from all bacterial spores, the spores of this bacterium Bacillus Astrophaeus are the most resistant to ozone. Use was made of standard spore strips with the qualification 10E6; these are strips on which in the order of 10⁶ bacterial spores have been deposited. The used strips were obtained from the company Etigam BV in Apeldoorn, Netherlands. According to statement of the supplier, these spores were obtained from the American Type Culture Collection (ATCC) 9372.

In this experiment, the spore strips were submitted to a sterilization process, wherein the ozone concentration, the degree of humidity and the treatment time were measured. The spore strips were removed from the sterilization chamber, and than were stored for 48 hours at a temperature of 37° C. in a test tube filled with tryptone soya broth (“tryptone Soya Broth”, TSB). TSB is a standard nutrition medium, obtainable at Tritium-Microbiologie BV in Veldhoven, Netherlands, type indication T406.24.0005.

Each time, four spore strips were treated simultaneously. After the storage time of 48 hours, for each spore strip it was investigated whether bacterial spores had grown. If visually no bacterial growth could be detected, it was assumed that all spores of the relevant spore strip had been killed. If visually bacterial growth could be detected, the relevant spore strip was qualified as “insufficiently sterilized”. The results are presented in the FIGS. 3A and 3B, wherein FIG. 3A relates to measurements at an ambient temperature of 20° C. during the sterilization process and FIG. 3B relates to measurements at an ambient temperature of 30° C. during the sterilization process. The horizontal axis in the figures represents the measured degree of humidity in percentages, the vertical axis represents the time integral of the ozone concentration, indicated as ∫[O₃], in units of gr·s/l. In the area 131, 100% of the bacterial spores of all four the test strips were always killed. In the area 132, each time one spore strip was insufficiently sterilized. In the area 133, each time two spore strips were insufficiently sterilized. In the area 134, each time three or four spore strips were insufficiently sterilized. The rectangular areas that are cut away from the top left of the graphs are areas belonging to test strips that were not analyzed.

It is noted that the sterilization results are in reality better than the figures suggest, because in the these measurements no distinction was made between a spore strip wherein not one spore was killed and a spore strip wherein just one single spore had survived the treatment.

It appears from the figures that an increase of the ambient temperature to 30° C. has a favourable effect. However, the ambient temperature may not become too high, because then the degradation of ozone is accelerated.

Furthermore the importance of a sufficiently high degree of humidity appears from the figures: at less than 50% RH no good sterilization proved to be possible. Surprisingly, however, it appeared that, in contradiction to the teaching in said publication, increasing the degree of humidity to 95% in general did not give clear improvement; FIG. 3A even suggests an optimal result at approximately 80% RH.

In principle, it is possible to further refine these measurements, and in particular to further investigate the influence of degree of humidity and ambient temperature in order to take these into account in the final sterilization process. However, this makes the sterilization process more complex. The present invention suggests to monitor the degree of humidity and to reject the sterilization process if the degree of humidity drops below 60% RH. Furthermore, the present invention proposes to apply as a minimum value for the ozone performance equivalent OPEQ a value that, as appears from the measurement results of FIGS. 3A and 3B, at all values of the degree of humidity above 60% RH leads to 100% sterilization, both at 20° C. and at 30° C., wherein it is noted that in practice the ambient temperature will usually be between 20° C. and 30° C. If desired, it is possible to prevent the start of the sterilization device if the ambient temperature is below 20° C., and if desired it is possible to provide the sterilization process with heating means to bring and to keep the ambient temperature above 20° C. Although it is thus preferred that the degree of humidity is approximately equal to 80% RH and that the ambient temperature is approximately equal to 30° C., the minimum value of the OPEQ is chosen based on a worst case situation, namely 60% RH and 20° C. A suitable value for OPEQ then is 15 gr·s/l. In the FIGS. 3A and 3B, this minimum value is indicated by the horizontal line 135. The present invention proposes to use this minimum value as the stop criteria for the sterilization process; it may be clear though that it is possible to execute the sterilization process during a longer time or at higher ozone concentrations, which will yield results above this horizontal line 135 in the FIGS. 3A and 3B, but this has no useful effect because the sterilization is already completed.

On the basis of the above-presented measurement results, the present invention thus proposes to control a sterilization process such that the ozone performance equivalent OPEQ is always respected as minimum value. This is illustrated by means of FIG. 4, which shows a graph of ozone concentration as function of the time, and FIG. 5, which shows a flow diagram of the process.

A sterilization process begins with a preparing phase, in which the instruments to be sterilized are introduced into the chamber 10, and in which the chamber 10 is possibly evacuated. Then a desired atmosphere is established in the chamber 10, with a pressure in the order of approximately 100 mbar below atmospheric pressure. The fan 30 is turned on (see step 201) to circulate the gas in the circuit 20. The moisture producing device 70 is turned on to increase the moisture content in the gas to minimally 60% RH, preferably approximately 80%. The ozone generator 60 is turned on to increase the ozone content in the gas. FIG. 4 illustrates that the process starts at time t₀, and that the ozone concentration OC is initially low, but rises to a mainly constant value. FIG. 4 furthermore illustrates that during the process the ozone content does not need to be exactly constant but may fluctuate.

During the process, with regular time intervals Δt, for example 1 time per second, the controller 90 receives the ozone concentration measured by an ozone detector of the sensor 40 and the moisture content measured by a moisture detector of the sensor 40. De controller 90 is designed to multiply the measured ozone concentration OC with the time interval Δt concerned, and to add the outcome OC·Δt in a memory M (step 203), until the value in that memory matches a value registered in the memory beforehand, corresponding to the ozone performance equivalent OPEQ (step 204). At that moment, the controller 90 is satisfied that the sterilization process has taken long enough to eliminate with certainty all bacterial spores possibly present. The controller 90 may now switch off the ozone generator 60 (step 205), which in FIG. 4 is illustrated at time t₆.

Thus, the controller 90 in fact calculates the time integral of the ozone concentration, which in FIG. 4 is illustrated by the hatching below the curve. FIG. 2 teaches that a temporary decrease of the ozone concentration compared to the target value is not harmful as such, but may be compensated by a correspondingly longer process time, and this is effectively reached by comparing the time integral of the ozone concentration with the value of the ozone performance equivalent determined from experiments.

The controller 90 may calculate the time integral over the entire process time from time t₀, which means for all values of the ozone concentration. In that case also very low values of the ozone concentration contribute to the time integral, while there is a good possibility that at these values there is hardly any contribution to the elimination of bacterial spores. Therefore, to increase the certainty, the controller 90 preferably takes into account a concentration threshold OCmin, which in the illustrated example amounts to 0.2 mgr/l: as long as the ozone concentration OC is lower than this threshold OCmin, the controller 90 does not take this concentration along in the calculation of the time integral (Step 211), which in FIG. 4 is illustrated by the white surface below the curve from time t₀ till time t₁, the time that the ozone concentration reaches the threshold.

FIG. 4 shows that it is also possible that, during the sterilization process, the ozone concentration drops below the mentioned threshold for some time; FIG. 4 illustrates this from time t₂ till time t₃. Also in that case, just to be sure, it can be chosen not to take the concerned measurement values along in the calculation of the time integral (step 211).

Also the opposite is possible, i.e. that during the sterilization process the ozone concentration surpasses a predetermined maximum value OCmax (4 mgr/l in this example); FIG. 4 illustrates this from time t₄ till time t₅′. In itself it is favourable that the ozone concentration is so high: it will only be in favour of the killing of bacterial spores. It is conceivable though that the validity of the formula OC·t=OPEQ is not guaranteed or checked for ozone concentrations above OCmax. In that case, in an embodiment of the invention, not the momentarily high value OC of the ozone concentration is used for the calculation of the time integral, but the mentioned maximum value OCmax (steps 212, 213), which in FIG. 4 is illustrated by the lacking of hatching above the horizontal line at 4 mgr/l.

It is noted that higher ozone concentrations will not be disadvantageous for the sterilization process, on the contrary, but within the context of the present invention it is considered disadvantageous that for this purpose pure oxygen and strong ozone generators are needed, for which reason the preference is given not to pursue a particular high ozone concentration; particularly, in contrast with the state of the art, an ozone concentration higher than 40 mgr/l is not pursued.

It will be clear for a person skilled in the art that the invention is not limited to the exemplary embodiments discussed in the preceding, but that several variations and modifications are possible within the protective scope of the invention as defined in the attached claims.

1. Method for sterilizing objects with ozone comprising:
positioning the objects in a sterilization chamber;
effecting an ozone-containing gas atmosphere in the sterilization chamber;
making the ozone-containing gas circulate via a circulation pipe outside the sterilization room, wherein the circulation pipe is provided with an ozone generator and a water mouthpiece;
establishing in the gas an air humidity in a range from approximately 60% to approximately 90%;
establishing in the gas an ozone concentration higher than 0.2 mgr/l;
defining in advance an ozone performance equivalent;
measuring the ozone concentration during the sterilization process on certain measurement moments; and
calculating the time integral of the measured ozone concentration;
wherein the sterilization process is continued until the time integral of the measured ozone concentration is minimally equal to the predetermined ozone performance equivalent.

2. Method according to claim 1, wherein the gas in the circulation pipe is moving with a velocity of approximately 8 m/s or more.

3. Method according to claim 1, wherein the gas in the sterilization chamber has a velocity in the range from approximately 0.25 m/s to approximately 0.35 m/s.

4. Method according to claim 1, wherein the gas in the sterilization chamber has an underpressure in a range from approximately 50 mbar to approximately 150 mbar.

5. Method according to claim 1, wherein the gas is circulated in the circulation pipe by a fan placed directly after an output opening of the chamber.

6. Method according to claim 1, wherein a sensor for measuring the ozone concentration is arranged in the circulation pipe, between the outlet opening of the chamber and the ozone generator.

7. Method according to claim 1, wherein the sterilization process is stopped at the moment when the time integral of the measured ozone concentration is minimally equal to the predetermined ozone performance equivalent.

8. Method according to claim 1, wherein the time integral of the measured ozone concentration is calculated in a memory by executing a running summation of Σ(OC·Δt), wherein Δt is the time distance between two neighbouring measurement moments, and OC is the ozone concentration.

9. Method according to claim 8, wherein the measured ozone concentration is compared with a minimum value, and wherein the summation is skipped if the measured ozone concentration is lower than the minimum value.

10. Method according to claim 1, wherein the ozone performance equivalent is approximately equal to 15 gr·s/l.

11. Method according to claim 1, wherein the gas has an air humidity of approximately 80%.

12. Method according to claim 1, wherein the gas has an ozone concentration in a range from approximately 2 mgr/l to approximately 40 mgr/l.

13. Method according to claim 3, wherein the gas in the sterilization chamber has a velocity of approximately 0.3 m/s.

14. Method according to claim 4, wherein the gas in the sterilization chamber has an underpressure of approximately 100 mbar below atmospheric pressure.
